(19) United States
(12) Patent Application Publication
(10) Pub. No.: US 2011/0177170 A1
(43) Pub. Date: Jul. 21, 2011

(54) IMPLANTABLE NEUROENDOPROSTHETIC SYSTEM, A METHOD OF PRODUCTION THEREOF AND A METHOD OF RECONSTRUCTIVE NEUROSURGICAL OPERATION

(76) Inventors: Andrey S. Bryukhovetskiy, Moscow (RU); Viktor I. Sevastianov, Moscow (RU)

(21) Appl. No.: 13/121,069

(22) PCT Filed: Feb. 13, 2009
(86) PCT No.: PCT/RU09/00067

§ 371 Date: Mar. 25, 2011

(30) Foreign Application Priority Data
Sep. 25, 2008 (RU) 2008138161

(51) Int. Cl. A61K 9/00; A61K 35/12; A61P 25/00

(52) U.S. Cl. 424/484; 424/93.7

(57) ABSTRACT

Intended for the use in neurosurgery and organ tissue engineering to replace defects of nervous tissue of a mammal brain and spinal cord (B&SC) in reconstructive neurosurgical operations, a tissue-replaceable artificial cell-biopolymer neuroendoprosthetic system (ACBP NEPS) for surgical plasty of defects of nervous tissue of B&SC, stimulating regeneration and growth of damaged axons of neural cells, comprises an elastic cell-biopolymer biologically active mass, produced from a heterogeneous collagen-containing matrix for implantation, and a biocomposition of cell preparations of various types of autologous cells of a patient. Also disclosed are a method of producing the ACBP NEPS, which provides for perfusion of the biocomposition of the cell preparations of various types of autologous cells of a patient into a heterogeneous collagen-containing matrix for implantation, and a method of reconstructive neurosurgical operation comprising implantation of the ACBP NEPS into a defect of neural tissue.

### CROSS-REFERENCE TO RELATED APPLICATIONS

This application is a U.S. National phase application of the International application WO 2010/036141 A1 (PCT/RU2009/000067) and claims priority to application 2008138161 filed on Sep. 25, 2008, in the Russian Federation, both applications being hereby incorporated by reference in their entirety.

### BACKGROUND OF THE INVENTION

1. Field of the Invention

The invention relates to the field of neurosurgery and tissue engineering of organs and can be applied for transubstantiation of defects of neural tissue of brain and spinal cord in reconstructive and repairing treatment of consequences of traumatic, ischemic injuries, and for surgical operations on central nervous system (CNS) and vegetative nervous system (VNS) of a mammal, for example a human. A list of the abbreviations used in the disclosure is placed in paragraph [0117] below.

2. Description of Related Art

Till the end of 20^{th} century, prosthesis of nervous tissue defects was considered impossible and almost unsolvable that can be explained by the dogmatic understanding of limited restoration capacities of nervous tissue by brain researchers, neurologists and neurosurgeons, as well as by dogmatic notion established in 1801 by Santiago Ramon-y-Cajal that neural cells were inherently unable to regenerate after the injury. However, the last decade of 20^{th} century has considerably changed the approach by accumulation of new scientific evidence of regenerative potential of CNS, reparative properties of neural stem cells (NSC) and conclusive proofs of restoration opportunities of the axons of injured neurons.

Known in the art is a preparation of hematopoietic stem cells (HSC), which is autologous HSC obtained from a patient peripheral blood enriched with cells containing CD34 antigen in the final concentration of (40 to 100)·10⁶ cells/ml. A therapeutic treatment of a brain and spinal cord (SC) traumatic disease is performed by intrathecal or intraventricular administration of the cell preparation to a patient (RU 2283119 C1, A61K35/14, 2006). However, the preparation consisting of solely HSC appeared insufficiently effective in the therapy of brain and SC nervous tissue defects.

Known is a biopolymer prosthesis <<NeuroGel<<™ to fill defects of nervous tissue (S. Woerly, V. D. Doan, F. Evans-Martin, C. G. Paramore, J. D. Peduzzi, “Spinal cord reconstruction using NeuroGel™ implants and functional recovery after chronic injury”, J. of Neuroscience Res. 2001. Vol. 66, P. 1187-1197). Research proved a possibility of injured axons to grow through biopolymer composition in a mammal SC and restoration of lost brain functions, while stem cells provide favorable conditions for axon regeneration. However, application of the <<NeuroGel<<™ prosthesis proved inefficient in the treatment of brain and SC defects. Moreover, the <<NeuroGel<<™ composition contains agents, prohibited to be used in humans.

Also known in the art is a multipurpose heterogeneous collagen matrix for implantation, which is an elastic mass prepared of two collagen sources, one being the tissue of vertebrate animal of one class, and the second being the tissue of other class of animals. The matrix consists of two phases: a solid phase represented by microspheres of a mammal tissue collagen and a liquid one represented by a denatured bird tissue collagen. This heterogeneous collagen-containing matrix was proposed for restoration of injuries of soft tissues and organs by means of implantation (RU 2249462 C1, A61K38/39, 2005). The matrix is accepted as the prototype of the claimed implantable neuroendoprosthetic system. Implantation of this collagen matrix into a defect of nervous tissue has not activated regenerative potential of the artificial implant in full due to big size of microspheres (300 to 400 μm) and their hardness that led to mechanical damage and death of transplanted stem cells.

### BRIEF SUMMARY OF THE INVENTION

An object of the present invention is to provide a tissue replaceable artificial cell-biopolymer neuroendoprosthetic system (ACBP NEPS) for surgical plasty of nervous tissue defects of brain and SC, that will stimulate damaged neural cell axons regeneration and growth, as well as to provide a production method of this system and a method of reconstructive neurosurgical operation for the transubstantiation of the defects of brain and/or SC and/or a VNS in a mammal with this system hereinafter referred to as the implantable neuroendoprosthetic system or ACBP NEPS.

The above object is reached by that, according to the present invention, it is provided an implantable neuroendoprosthetic system for the transubstantiation of defects of a brain, spinal cord and vegetative nervous system in a mammal in reconstructive neurosurgical operations, wherein the system is an elastic cell-biopolymer biologically active mass produced from a heterogeneous collagen-containing matrix for implantation and a biocomposition of cell preparations of various types of autologous cells of a patient.

More specifically, said biocomposition comprises, in a NaCl solution, neural stem cells (NSC), neuroglial ensheathing cells (NGEC), endothelial cells with CD34+ marker (EC), and purified mononuclears (MN) in the following ratios (in parts according to numbers of the cells): 0.8 to 1.2 of NSC; 1.6 to 2.4 of NGEC; 4 to 6 of EC; 4000 to 6000 of MN. Preferably, 0.5 to 1.3% solution of NaCl is used. As the heterogeneous collagen matrix, a composition of a heterogeneous implantable gel Sphero®GEL is mainly used.

Said biocomposition can further comprise stimulators of cell regeneration, nerve growth factors and vascular growth factors. The most appropriate, efficient and safe (in consideration of swelling ratio of the Sphero®GEL matrix) is the ACBP NEPS in which numbers of said cells contained in 0.5 to 1 ml of 0.9% NaCl solution per 1 ml of said heterogeneous collagen-containing matrix are as follows: 10⁶ of NSC; 2·10⁶ of NGEC; 5·10⁶ of EC; 5·10⁹ of MN and which comprises 0.1 to 0.2 ml of standard solution of a cell regeneration stimulator (methyluracil, leucogen, ATP, pentoxyl etc.).

Said object is also reached by that it is provided a method of production of an implantable neuroendoprosthetic system for the transubstantiation of defects of a brain, spinal cord and vegetative nervous system in a mammal in reconstructive neurosurgical operations, comprising perfusion of a biocomposition of cell preparations of various types of autologous cells of a patient into a heterogeneous collagen-containing matrix for implantation to obtain an elastic cell-biopolymer biologically active mass.

Preferably, the perfusion is performed by centrifugation. The centrifugation is carried out within 1.5 to 2.5 minutes at 1,500 to 2,500 revolutions per minute. The biocomposition is prepared from cryopreserved cell preparations that, immediately before the production of said implantable neuroendoprosthetic system, are defrosted at a water bath at 37 to 40° C. and then washed at least twice in a physiological NaCl solution.

More specifically, said biocomposition comprises, in a NaCl solution, neural stem cells (NSC), neuroglial ensheathing cells (NGEC), endothelial cells with CD34+ marker (EC) and purified mononuclears (MN). A source of the NSC and the NGEC is preferably olfactory sheath of the nose of a patient, and a source of the EC and the MN is either a bone marrow of the patient or a leukoconcentrate of mobilized autologous stem cells of the patient, wherein the leukoconcentrate is obtained during separation of the patient's peripheral blood after patient stimulation with a granulocyte colony-stimulating factor. Stimulators of tissue regeneration, nerve growth factors and vascular growth factors can be added in said biocomposition. The method of the ACBP NEPS production of the present invention is carried out in sterile conditions either directly in an operation room (ex tempore), or in a culture laboratory (period of implantation up to 6 hours).

Said object is also reached by that it is provided a method of a reconstructive neurosurgical operation to replace defects of a brain, spinal cord and/or vegetative nervous system in a mammal, including implantation of a neuroendoprosthetic system in the form of an elastic cell-biopolymer biologically active mass into a defect of neural tissue, wherein said mass is obtained from a heterogeneous collagen-containing matrix for implantation and a biocomposition of cell preparations of various types of autologous cells of a patient.

In more detail, said biocomposition comprises, in a NaCl solution, neural stem cells (NSC), neuroglial ensheathing cells (NGEC), endothelial cells with CD34+ marker (EC) and purified mononuclears (MN). A composition of a heterogeneous implantable gel Sphero®GEL is preferably used as said heterogeneous collagen-containing matrix. The neuroendoprosthetic system is implanted by its placing into the defect and by filling a whole volume of a cyst or a lesion of the brain and/or spinal cord with the system. After the neuroendoprosthetic system has been placed into the defect, the system is covered with an autologous muscle fascia or an artificial dura mater and/or a biodegradable synthetic polymer coat to reduce a contact of the neuroendoprosthetic system with cerebrospinal fluid (CSF) of the patient. An implantable biopolymer membrane ElastoPOB® is preferably used as said biodegradable synthetic polymer coat. In case of a complete anatomical break-up (neurotmesis) of the spinal cord, the neuroendoprosthetic system is implanted in the following way: a conduit is formed from an artificial arterial graft and the neuroendoprosthetic system by which, at least partially, said graft is filled, the conduit is then placed in a gap between ends of the injured spinal cord and then pia mater of distal and proximal ends of the injured spinal cord is sutured to the conduit walls. The length of the artificial arterial graft is equal to the length of said gap, and the width of the graft is equal to the diameter of the spinal cord in the lesion site. In an intramedullar or intracerebral implantation, the neuroendoprosthetic system is isolated from a direct impact of CSF.

The implantable biopolymer membrane ElastoPOB® is produced by ZAO Biomir-Servis (Moscow) according to technical standards TY 9398-002-54969743-2006, registration certificate No. ΦC01032006/5581-06 of Dec. 28, 2006.

### BRIEF DESCRIPTION OF DRAWINGS

Efficiency of the ACBP NEPS of the present invention is illustrated by the following figures:

FIG. 1 shows a diagram demonstrating efficiency of the treatment of patients with a traumatic brain and spinal cord injury by the method of the present invention and conventional method;

FIG. 2 shows urograms of a patient before (A) and after (B) implantation of the ACBP NEPS according to the present invention.

### DESCRIPTION OF PREFERRED EMBODIMENTS

Body and structure of the proposed ACBP NEPS provide adequate modeling of a neuroimplant in damaged regions of brain and spinal cord and local stimulation of regeneration processes in the brain and spinal cord lesion sites. Application of only autologous cells preparations in the ACBP NEPS averts problems of histocompatibility and immune conflict, as well as the need of using immunosuppressants. Ethic, legal and moral limitations are absent as the treatment involves only autologous cells of the patient. A risk of infectious and viral contamination is excluded, as well as a risk of prion infection possible in case of application of xenogenous materials. Exact number of the applied ACBP NEPS is defined depending on volume, character and location of a brain/spinal cord lesion.

The creation of the proposed ACBP NEPS was based on the development and production of a polymer matrix that would meet specific demands of “bridging” the gap between the damaged regions of brain/spinal cord, provide support and nutrition for the cell preparations, function as an orienting vector for the axons of the injured autoneurons and neuroglial cells, help stimulate regeneration of the injured axons, and, contributing to their growth through the defect region of the nervous tissue, prevent the process of sprouting from occurring. The proposed matrix should meet safety criteria and biodegrade into safe components within a preset time period (8 to 12 months) being replaced by restored nervous tissue.

The preferable matrix to be used in the proposed ACBP NEPS is the composition of the heterogeneous implantable gel traded as Sphero®GEL (Russian trademark registration No. 269774) and produced by ZAO Biomir-Servis (Moscow) according to technical standards TY 9398-001-54969743-2006 of Dec. 26, 2006, registration certificate No. ΦC 01032006/5580-06 of Dec. 28, 2006.

The Sphero®GEL matrix consists of microparticles of cross-linked collagen of type VII obtained from farm animals, which microparticles are suspended in elastic homogeneous gel. The matrix is a unique complex of peptides (30 to 50 mg/g), uronic acids (0.8 to 1.2 mg/g) and hexosamines (2.0 to 3.0 mg/g). Amino-acid composition of the matrix is identical to the collagen but its hexosamine content is twice as much, and uronic acids content is 15 times higher, than that in the collagen. The Sphero®GEL matrix is stable for long-term storage and not susceptible to syneresis (expulsion of liquid). In this condition, it does not interfuse with water and hydrophobic liquids. At 37° C., the gel turns into liquid interfusing with water and the microparticles of the cross-linked collagen in all ratios. The size of the gel microparticles can be varied from 30 to 300 μm. Swelling capacity of the Sphero®GEL matrix is no less than 87%, pH=4.8 to 7.2. Average time of bioresorption in a body varies from several weeks to 9 months depending on the site of implantation and size of the microparticles. High biological compatibility and stimulating properties of the biopolymer implant Sphero®GEL contributing to repairing processes in the site of tissue lesion have been experimentally and clinically confirmed. Preclinical and clinical trials were performed by the FGU Scientific Research Institute of Transplantology and Artificial Organs, ZAO NeuroVita Clinic, the Troitsk Hospital of the Russian Academy of Sciences, the Civil Aviation State Clinical Hospital, S. P. Botkin State Clinical Hospital.

The clinical trials have proved highly efficient functional properties of the Sphero®GEL matrix applied as:
biologically active artificial synovial fluid for therapeutical treatment of knee joints arthroses;
implants for surgical intervention of peripheral nerves conductance disorder;
implantable carriers for transplantation and holding stem cells for spinal cord injury treatment.

It was just the Sphero®GEL matrix that, after it had been tested on animal models of spinal cord injury in the ZAO NeuroVita Clinic for three years provided the basis to the proposed neuroendoprosthetic system. To establish a cell composition with the stimulators of tissue regeneration, further perfusion of a specific cell composition into the Sphero®GEL matrix was proposed in the course of experimental and clinical trial of the matrix. The main challenge of the ACBP NEPS development was to select basic components of the cell biocomposition and to determine their concentration in 1 ml of the biodegradable Sphero®GEL matrix.

Endothelial cells with the CD34+ marker and neural stem cells possess the property of target oriented migration to lesion sites. Transplanted cells form clusters of progenitor cells in the brain/spinal cord tissue. This phenomenon became essential in solving the problem of injured brain/spinal cord regeneration. However, only after long-term animal experiments and 32 surgeries on humans, the optimal combination of the biopolymer with the cell composition and the regeneration stimulators has been found, that has allowed to achieve maximal clinical and neurophysiological effect of surgical operations in tissue engineering of brain/spinal cord and resulted in the creation of the ACBP NEPS.

The ACBP NEPS is an artificial analogue of nervous tissue and contains all its basic cell elements (neurons, neuroglial cells, endotheliocytes, etc.) and a polymer matrix with specified parameters of biodegradation (from 8 to 12 months). Body and plasticity of the ACBP NEPS allow for adequate modeling of neuroimplant in damaged anatomical structures of the brain and also provide local stimulation of regeneration processes in the lesion site. The ACBP NEPS is able to fulfill bridging and nourishing functions to facilitate growth of injured axons through pathologically modified regions of brain/spinal cord tissue.

NSC contained in the biocomposition of the proposed ACBP NEPS can reconstruct injured nervous tissue and first of all provide the restoration of gray matter of the spinal cord, while NGEC can stimulate differentiation of blood precursor cells into astrocytes and oligodendrocytes that can restore white matter and be a source of the cells remyelinizing damaged axons. Adding hematopoietic stem cells (CD34+CD45−) oriented for endotheliocytic differentiation, hence, presenting the source of EC, to the biocomposition enhances development of new vessels, leads to rapid formation of a microcapillary vascular network in the endoprosthetis, improves local blood supply. The use, as a component of the cell biocomposition, of non-hematopoietic stem cells being in the peripheral blood mononuclear fraction (MN) after stimulation by colony-stimulating factors leads to the production of a large amount of growth factors that significantly contribute to axon regeneration.

Practical application of various cell preparations in the Sphero®GEL matrix in clinical practice has showed that the best clinical and neurophysiological outcomes are achieved from specific ratio of certain cell preparations in the Sphero®GEL matrix, rather than from isolated application of the matrix and intracerebral or intramedullar administration of these cell cultures, thus resulting in creating specific artificial microenvironment for the damaged axons, close to that in natural tissue of developing brain/spinal cord by its cell structure.

Production of cultured NGEC and NSC suspensions follows a standard method. The source of these cells is a fragment of olfactory sheath of the patient's nose, which fragment is isolated in the course of otolaryngologic endoscopic surgical manipulation. Preparations of the hematopoietic stem cells, which are the source of EC, as well as non-hematopoietic stem cells residing in the mononuclear fraction (MN) are produced according to the technique described in the RU patent No. 2283119, incorporated herein by reference, and in the registration certificate of Federal Service on Surveillance in Healthcare and Social Development No. ΦC-2006-/151 of Jul. 1, 2006.

The proposed method of ACBP NEPS production and the proposed method of neurosurgical operation can be implemented only under conditions of neurosurgical operation theatre of a surgical department and anesthesiology and resuscitation department of a multi-field hospital licensed for medical activities involving high technologies in neurology, neurosurgery, hematopoietic stem cells harvest and application of cell technologies by neurosurgeons and anesthesiologist-resuscitators trained for the use of cell technologies. The hospital should be equipped with present-day diagnostic means (MRI, spiral computer tomography, angiographic equipment, cytofluorometer, blood separator) or have agreements with institutions equipped with these means. A laboratory certifying autologous hematopoietic stem cells of peripheral blood (AHSCPB), NSC, NGEC and EC must be licensed as a laboratory authorized for cell techniques application, meet the GLP (Good Laboratory Practice) standard and be able to perform complete analysis of a cell preparation in the scope recommended by the European Bone Marrow Transplant Registry. The laboratory must be capable of testing a biopreparation for sterility, toxicity, and of culturing the material, i.e. be equipped with a sterile box with a laminar safety cabinet, CO₂-incubator, bifocal microscope and a kit for culturing. The personnel must be additionally trained in transfusiology.

The procedure of use of the proposed ACBP NEPS consists of several consecutive stages:

I—specific examination of a patient;

II—harvest and preparation of biomaterial (including the proposed method of the ACBP NEPS production) and

III—reconstructive neurosurgical operation on the spinal cord or brain with intraoperative preparation of the ACBP NEPS and its implantation.

### Stage I. Specific Patient Examination

At this stage, the requisite clinical and paraclinical examination of a patient according to standard protocol is performed. Diagnostic parameters of the site of lesion are analyzed, the strategy of tissue engineering for brain or spinal cord is developed, indications and contraindications for ACBP NEPS implantation are established, as well as the time schedule for every stage adjusted for the results of examination. If necessary, cell transplantation is mathematically modeled and the volume of the cell preparation to prepare the ACBP NEPS according to MRI data is estimated. This stage must not be formal testing and examination. It is at this very stage where absolute and relative contraindications are determined, as well as medical and social prognosis of the surgery in tissue engineering on the brain and spinal cord for the patient. Inasmuch as the intramedullar and/or intracerebral introduction of NCS and NGEC preparations is planned, main attention should be given to immunochemical characteristic of blood and CSF of the patient. Additional introduction of neurospecific proteins during the transplantation of neurons and neuroglia-containing cells in the course of the surgery, combined with initially high value of neurospecific antigens in the patient's blood and CSF can further deepen self-destructive process in the CNS. At the same time, introduction of neurospecific proteins during transplantation of neurons and neuroglia-containing cells in the course of the surgery, combined with the initial presence of antibodies to neurospecific antigens in the patient's blood and CSF can trigger or exacerbate the autoimmune process, and in certain cases the autoimmune lysis of injured tissue. The patients with antibodies to neurospecific proteins and immunological deficit are not recommended as candidates for such surgeries.

### Stage II. Harvest and Preparation of Biomaterial

### II.1 Composition of the Heterogeneous Implantable Gel Sphero®GEL

The Sphero®GEL composition is produced according to the method described in the mentioned RU patent No. 2283119 incorporated herein by reference. It is produced in injection formulation in syringes of 1, 2 and 5 ml.

At room temperature, the heterogeneous matrix Sphero®GEL is an elastic mass stable at long storage, not susceptible to syneresis (separating out of liquid). At the temperature of 37° C., viscosity of the biopolymer matrix sharply reduces due to a weakly cross linked liquid component, while the physical condition of a globular (solid) component does not change. Immunogenicity of the obtained matrix is sufficiently low.

Antibiotics, antiseptics, stimulators of regeneration and anticoagulants can be introduced into the matrix immediately before surgical intervention as additional components. The antibiotics can include penicillins (such as benzylpenicillin, cloxacillin, ampicillin), cephalosporins (such as cephaloridin, cefuroxime, cefotetan, ceftazidime), carbapenems (such as meropenem), monobactams (such as aztreonam), aminoglycosides (such as streptomycin, gentamicin, amikacin), tetracyclines (such as tetracycline, minicyclin), macrolides (such as erythromycin, azithromycin), lincosamides (such as lincomycin), antibiotics of the peptide group (such as polymyxin B, polymyxin M, ristomycin, bacitracin). As the stimulators of cell regeneration, methyluracil, leucogen, adenosine triphosphate acid (ATP), pentoxyl, potassium orotate, inosine, etadenum can be used, for example.

Antibacterial and antiviral components, as well as antiaggregational agents can be additionally introduced into the Sphero®GEL composition. The stimulators of cell regeneration are perfused into the Sphero®GEL directly in a surgical room in the volume not exceeding 0.2 μl per 1 ml of the matrix.

The Sphero®GEL matrix introduced directly in the injury site is in time replaced by initial tissue with no scar formation.

Advantages of the Sphero®GEL matrix used for implantation in injured brain and spinal cord are as follows:
multifunctionality (supporting and trophic functions for cell cultures and stimulation of axon regeneration and neovascularization);
high biocompatibility of the final product, as well as of the products of its biodegradation, on protein and cell levels;
capability to stimulate proliferation and differentiation of neural cells;
controllable period of biodegradation varying from several weeks to several months (the final products of the biodegradation are water and carbon dioxide);
cavitation capacity at the immediate contact with biological media, that provides for neovascularization);
possibility of sterilization without changing medical and biological properties.

### II.2 Processing and Production of the Biocomposition of Cell Preparations

### II.2.1 Production of Stem Cells

The procedure can be conditionally subdivided into two stages, namely mobilization of stem cells into peripheral blood and stem cell harvest.

### II2.1.1 Mobilization of Stem Cells into Peripheral Blood

To increase the number of stem cells in peripheral blood, a donor gets 8 subcutaneous injections of granulocyte colony-stimulating factor (G-CSF) every 10 to 12 hours for 4 days. G-CSF is a pharmaceutical obtained by gene engineering and absolute analogue to human factor. The first three days, the dose makes 2.5 μg/kg and the last day, the dose is doubled. The blood is tested daily and ultrasound examination of abdomen is to be done at day 4 to 5.

### II.2.1.2 Stem Cells Harvest

Stem cells are harvested on day 5 since the G-CSF stimulation has started in a blood separator of COBE-spectra type using a disposable system for separation and standard solutions. The procedure lasts 3 to 4 hours depending on the speed of the procedure, weight of the patient and blood test results. In the course of the procedure, blood is sampled from a vein, processed inside the separator, the stem cells are sampled and the rest components of blood are returned to the donor through another vein. Veins are accessed by the puncture of two peripheral veins or by dual-lumen central catheter inserted in the subclavian vein for the séance. Average volume of the obtained material varies from 300 to 400 ml. The gathered material is evaluated by two parameters: according to the total number of nuclear cells (NC) in the sediment and according to the number of CD34+ cells per every kilogram of the patient's weight. NC in sediment are determined by counting in Gorjaev's chamber before any manipulations. The percentage of the CD34+ in the cell preparation obtained in the course of cytapheresis is determined by a flow cytometry method.

### II.2.2 Specification of Peripheral Stem Cells

A subpopulation composition of the CD34+ is determined by cytofluorometry with the method of triple-labeling (simultaneous staining of the cells with antibodies, loaded with various dyes, to three different antigens).

### II.2.3 Standardization of the Preparation

The number of precursor cells is determined by cytofluorometry in a direct immunofluorescence test (DIT).

A method of double labeling is used with simultaneous staining of cell substrate with monoclonal antibodies (MCA) to a CD34 antigen, that is the main marker of a hematopoietic stem cells pool, and to a CD45 molecule that is a common leukocyte antigen characteristic for all hematopoietic stem cells. Such method permits direct calculation of the ratio of CD34+ cells and all hematopoietic (CD45+) cells in the product.

To evaluate non-specific binding levels, a part of the cells is stained with isotypic controls such as conventional mouse immunoglobulins IgG1 of isotype (IgG1), labeled with dyes analogous to the label of the monoclonal antibodies (PE, FITC, PerCP) in use.

### II.2.4 Preparation of Cell Tests

Before the reaction, the cells of peripheral blood and cytapheretic product are cleared from erythrocytes by a standard lysis method and further washing in buffered saline with bovine serum albumin (BS-BSA) by centrifuging at 1,000 g for 5 minutes. BS-BSA can be replaced with the TC Hanks solution or TC Medium 199.

### Method of Lysis of Erythrocytes:

1. 2 ml of the lysing solution is added to 0.2 to 0.5 ml of the cell sediment, mixed and incubated till the solution is clear (laked).

2. The cells are washed twice by the 199 medium in a centrifuge (1000 g, 5 to 7 min).

The selected cells are subjected to the DIT in a 96-socket plate, a plate with the following 3 sockets being used to count peripheral HSC in any product:
Unstained cells;
Cells stained by isotypic controls labeled according to the labels of the MCA used;
Cells stained by the MCA both to the antigens CD34 and CD45.

It should be especially noted that MCA to CD34 were preferably phycoerythrin (PE)- or peridin chlorophyll (PCP)-labeled, since these fluorochrome data have a higher level of specific signal as compared with fluorescein isothiocyanate (FITC).

Optimal to count CD34+ cells are; antibodies to CD34 of HPCA-2(8G12) clone, isotype IgG1.

Thus, the reference panel is the following:

1. IgG1 PE control+IgG1 FITC control;

2. IgG1 PE control+MCA to CD45 FITC;

3. MCA to CD 34 PE (HPCA-2)+MCA to CD45 FITC.

### II.2.5 DIT Testing

1. No less than 500,000 cells per a socket are inserted into the sockets.

2. Further, a cocktail of antibodies according to the reference panel is introduced into each socket and carefully resuspended by a pipette. 10 μl of each MCA per a socket is taken, the total number of MCA in the socket being 20 μl.

3. The cells are incubated with antibodies for 30 minutes at 4° C. (a bottom shelf of a conventional refrigerator).

4. Incubation over, the cells are twice washed from unconjugated antibodies by the 1,000 g centrifugation for 5 to 7 minutes.

5. The cells are put into special plastic tubes to count on a flow cytometer.

6. The volume of cell suspension in every tube is brought to 200 to 500 μl by adding PBS-BSA.

The count should be done immediately after the testing.

### II.2.6 Count and Record on Flow Cytofluorometer

Reaction is assessed on a flow 5-parameters cytometer. The scheme is applicable for a flow cytofluorometer of any configuration.

The CD34+ cells in peripheral blood represent a minor cell population. Even under the condition of preliminary hematopoietic stimulation, the maximal percentage of these cells is 1.0 to 3.0%. Therefore, to count these cells, no less than 20,000 cell events have to be accumulated in each analyzed sample.

Cell collection and analysis is performed in the gate of CD45+ cells, which includes all hematopoietic cells, the gate herein being understood as the event accumulation region restricted by certain parameters. In this case, by SSC/FL-1(CD45FITC), i.e. the abscissa axis of a dot cytogram will show all CD45+ events. On the ordinate axis (SSC parameter—side light scattering) the cell events will be located according to their granularity (a cytometric term, not a morphologic one).

The count of the absolute number of CD34+ in 1 μl of blood and of cytoconcentrate was fulfilled on the baseline of the number of leukocytes in a hemogram on the day of testing.

### II.2.7 Principle of Detecting CD34+ Cells in Hematopoietic Tissue. Recording Samples

1. Region of analysis/gate selection. Menu of cell samples registration Acquisition is open on the appliance. At the first stage in a setup mode (a mode of cell sample review without recording), samples No. 1 (IgG1PE+, IgG1FITC) and No. 2 (IgG1PE+CD45 FITC) are viewed and CD45+ events are detected. These events are located to the right from 10¹ values (average threshold value of specific signal level for FITC fluorescein) on the x-axis—FL-1 (CD45+ cells) and are clearly visualized in comparison with the control sample No. 1.

According to sample No. 1, the entire region located to the right from major cell density is confined, that is gate that includes only CD45+ events in sample No. 2 and contains minimum of cell events in sample No. 1.

2. The appliance is switched to recording samples mode (Normal) and sample No. 1 is collected and recorded per 20,000 cell events without a gate (the whole cell population). Recording of the sample in the gate is impractical as MCA to CD45 antigen are absent in it. As shown above, the sample is required for the correct selection of the gate containing only CD45+ events.

3. Samples Nos. 2 and 3 are collected and registered in gate CD45+. The gate is identical for both sample No. 2 and sample No. 3, i.e. gate parameters are not changed during sampling. Minimum of cell events equals 20,000 for each sample.

### II.2.8 Recorded Samples Analysis

1. The appliance is switched to the menu of recorded samples analysis (Analysis). Then sample No. 2 cytogram (dot-plot) is opened in SSC/FL-2 parameters, in R1 gate—CD45+ (the gate was chosen previously when recording collection and was automatically transferred to the analysis mode). Thus, the y-axis will display SSC parameter, i.e. granularity of events in the analyzed gate, and the x-axis displays FL-2 that is the detector reflecting levels of nonspecific binding for phycoerythrin stain, which levels are detected by antibodies to mouse immunoglobulin IgG1PE.

2. The control marker is set on the received cytogram, so that almost no cell events appear in the right low quadrant. In average, the values for the vertical bar of the marker will make about 130 (to the right from 10²), and for the horizontal marker bar, the average values will be 60 that corresponds to SSC-low, i.e. the cells with minimum cell inclusions. Therefore, the levels of nonspecific binding will be equal or close to zero.

3. Automatically switch to sample No. 3. All indications of the appliance for sample No/2 are saved, i.e. cytogram is open in SSC/FL-2 parameters, the gate chosen earlier during the collection on the CD45+ cell events is saved and the meanings of the control marker set on sample No. 2 are saved too. However, on this cytogram, the x-axis displays specific CD45+ events rather than the levels of non-specific binding as in the second sample. CD45+ percentage is displayed by the appliance automatically.

### II.2.9 AHSCPB Cryopreservation

### II.2.9.1 AHSCPB Fraction Isolation

Separated cells are concentrated by centrifuging at 2,000 revolutions per minute speed for 10 min at +18° C. Plasma is maximally removed from the container by a manual plasmaextractor, the remaining cell volume being of 40 to 60 ml.

### II.2.9.2 Adding Cryoprotector

Purified dimethyl sulphoxide (DMSO) is used as a cryoprotector of hematopoietic stem cells. Equal volume of polyglucin with DMSO is added to the obtained cells at continuous stirring. DMSO reacts with polyglucin exothermally with moderate heat liberation. DMSO concentration in polyglucin is 10 to 12%. Thus, its final concentration in the material being frozen will make 5 to 6%.

Application of polyglucin permits to use half as much of the DMSO amount, up to 5 to 6% in the final concentration because polyglucin can disaggregate cells thus improving penetration of cryophylactic into the cells, moreover, polyglucin (6% dextran) is a cryophylactic per se.

### II.2.9.3 Cell Count

The next stage necessarily involves counting of nuclear cells as well as CD34+ cells to be frozen.

### II.2.9.4 Choice of Optimal Volume of Frozen Material

To provide the best conditions for cryopreservation, a correlation of plastic container volume and volume of the material to be frozen therein and concentration of frozen cells are important. It has been determined that the optimal concentration of the frozen cells is from 40·10⁶ to 100·10⁶ 1 cells per ml.

### II.2.9.5 Stem Cell Freezing

Depending on the final volume of material to be frozen, the number of polymer tubes (15 to 20) for deep freezing should be chosen. Then the cell suspension is placed into a container for cryopreservation.

To freeze the tubes with cell suspension, they are put into a programmed freezer or a laminated plywood container (10 mm of total thickness) with a tight lid and of the size consistent with the size of the tubes. Then the container with the material to be frozen is put into liquid nitrogen vapor at minus 165 to minus 170° C.

The speed of cooling of the material at the temperature from 0° C. to minus 40° C. is 1.1 degree per minute. This cryopreservation method levels crystallization plateau that usually is clearly seen at program freezing in an electronic device, and the composition of the frozen material remains unchanged when stored.

In one or two hours after the beginning of freezing, the container can be transported to a warehouse where it will be stored in liquid nitrogen or its vapor till transplantation.

### II.2.10 Preparing AHSCPB to Administration. Defrosting Hemopoietic Stem Cells

The product is defrosted immediately before transplantation in 37 to 40° C. water bath till the moment of transition of the frozen product into a liquid state. Then the mobilized hemopoietic stem cells are deposited by centrifugation at 1,500 rev/min to the bottom of a centrifugal plug, the supernatant is drained, and 1 ml of 0.9% NaCl physiological solution is added. The procedure is repeated twice. The product of defrosted autologous stem cells can be administered within 6 hours after its preparation. If the product was not used during this time, it must be recycled.

### II.2.11 Taking NSC and NGEC

Nasal mucosa has to be endoscopically taken with informed consent of a patient. Nasal cavity was subjected to vasoconstriction by 0.1% solution of Xylometazoline. Under contact anesthesia with 10% solution of Lidocaine and local anesthesia with 1% solution of Procaine, a 3×4 mm size fragment of an upper portion of nasal septum mucosa is exsected. Bleeding is suppressed by cotton plugs with solution of Xylometazoline and hydrogen dioxide. The plug is removed 24 hours after the surgery in the absence of any nosebleed. In case of even slight bleeding, the nasal passages are repeatedly plugged for 24 hours.

### II.2.12 Method of Obtaining and Culturing NGEC Olfactory Epithelium (Ensheathing Cells)

Olfactory epithelial tissue was sampled from the upper third of superior nasal meatus of adult patients. Obtained tissue was washed in Hanks solution that contained antibiotics (Streptomycin, Penicillin) and antimycotic (Amphotericin). The tissue sample was minced and incubated in a Trypsin (0.05%) and EDTA (0.02%) solution (40 min, 36.5° C.). The tissue was dissociated by multiple pipetting, centrifuged and sedimented cells were washed with Hanks solution with 5% serum. The total number of cells in the suspension and percentage of viable cells were determined in a Gorjaev's count chamber by trypan blue staining. The cells (whose final concentration was 100,000 cells per ml) were cultured in 12-well trays on a polylysine/laminin substratum for 10 to 15 days (5% CO₂, 36.5° C.) in the following medium: DMEM/F12 (Gibco), fetal calf serum 10% (Gibco), glutamine 2 mM (Gibco), glucose 0.8%, mixture of insulin, transferrin and sodium selenite (Gibco, 1:100), HEPES buffer (10 mM), human neuregulin-1 β-1/heregulin 1-β-1 EGF domain 2 ng/ml (R&D systems). Medium was changed every 3 to 4 days. Dense monolayer having being developed, the cells were removed for subsequent passages from substratum by trypsin/EDTA solution, washed with Hanks solution and reseeded into flasks with polylysine/laminin substratum (25 cm², 10,000 to 12,000 cells per cm²). After 3 to 4 passages the cells were removed from substratum by trypsin/EDTA solution, washed with Hanks solution and the obtained suspension was either used for transplantation or frozen with cryoprotector (10% serum, 90% EDTA) and stored in liquid nitrogen at minus 70° C. For transplantation, the cryopreserved cells were defrosted; their viability was detected by trypan blue staining. Control tests showed no less than 90% of cells retained viability after storage.

For cytochemical identification of olfactory epithelial glial cells, a part of suspension was cultured after every passage on a cover slip (22×22 mm) in Petri dishes for a week. Primary antibodies to glial fibrillary acidic protein (GFAP, 1:6; monoclonal antibodies obtained in the Immunochemical Laboratory of Serbsky's State Research Centre of Social and Forensic Psychiatry), nestin (Chemicon International, CA, 10 μg/ml) and low affinity nerve growth factor receptors (p 75, Chemicon International, Inc., 10 μg/ml) were used for immunocytochemical analysis. The obtained preparations were analyzed and photographed by fluorescent microscope Leica DLMB.

### II.2.13 Method of Isolation and Culturing of NSC of Olfactory Sheath of Patient's Nose

Tissue of olfactory sheath including olfactory epithelium and a layer of connective tissue (lamina propria) is isolated from patients with a spinal cord injury and processed according to standard culture protocol.

10 by 5 mm size fragments of mucosa dissected under local anesthesia from the upper part of superior nasal meatus were accepted for the research. The sampled tissue was delivered to a laboratory in cool Hanks solution without Ca²⁺ and Mg²⁺ (HBSS), containing antibiotic and antimycotic agents (1:100; Gibco). Delivery time does not exceed 2 hours. After repeated wash in the same solution, blood vessels were removed from mucosa, then the tissue was minced and incubated for 40 minutes at 36.5° C. in 0.25% trypsin/EDTA solution prepared on 0.01 M phosphate buffer (PBS, pH 7.4). Activity of ferments being blocked by DMEM medium (Gibco) that contained 3% of serum, the tissue was washed in three changes of Hanks balanced salt solution (HBSS, Sigma) and dissociated by repeated pipetting in a nutritive medium. Medium composition: 90% of minimum Eagle medium (MEM, Sigma), fetal bovine serum (FBS, Gibco, Invitrogen), 0.8% of glucose, 2 mM glutamine (Gibco), B27 supplement (Sigma), HEPES 20 mM, growth factors (only for primary cultures) namely fibroblast growth factor (FGF2, 1 ng/ml, Sigma), neural growth factor (NGF 2 ng/ml, Sigma).

The obtained cell suspension is centrifuged (3 minutes at 1200 revolutions per minute), the sediment is resuspended in a nutritive medium of the same composition.

The number and viability of the dissociated cells are checked in the Gorjaev's chamber after 0.1% trypan blue staining. Cell suspensions with 85 to 95% of viable cells are only used for further culturing.

The dissociated cells (5.10⁵ cells per ml) are cultured in 12-well trays on polylysine/laminin substratum for 14 days (36.5° C., 5% CO₂). One third of the nutritive medium is partially changed 2 times a week. Primary culture, after confluent cell monolayer is formed, is removed by trypsin/EDTA solution. The cells are resuspensed in the nutritive medium after washing in HBSS and centrifuging. The cell suspension (10,000 to 12,000 cells per cm²) is placed into 12-well trays or dishes (square of 25 cm²). In this way, the cultures are passed 4 times till the confluent monolayer has been formed. Free floating and attached to substratum neurospheres formed in the cell monolayer are selected by Pasteur pipettes and dissociated by the ferment processing method described above. The selection of the neurospheres permits to separate them from accessory glial cells, fibroblasts and stromal (foot) cells attached to the substratum. The cell suspension of the neurospheres, after washing and centrifuging, is resuspended in the nutritive medium and cultured in 12-well trays (10,000-12,000 cells per cm²) and on cover slides (18×18 mm) in Petri dishes till confluent monolayer has been formed. The obtained cultures are used for cytological and immunocytochemical tests. A part of the cells of last passages is frozen in cryopreservation medium (90% serum, 10% dimethyl sulfoxide) and stored in liquid nitrogen.

### II.2.14 Immunocytochemical Tests

The cell monolayer is fixated in 4% solution of paraformaldehyde prepared on 0.01 M phosphate buffer (pH 7.4) for 30 minutes. After PBS washing (3×10 min), the cells are incubated for 24 hours at 4° C. with primary antibodies to β-tubulin (1:300; Chemicon), nestin (1:100, Chemicon) and neuronal specific enolase (1:100, the antibodies are obtained in our laboratory). After PBS washing, the cells are successively processed by biotinylated antibodies with avidin-biotin complex (ABC, Vector Laboratories, Inc), and diaminobenzidine solution prepared on phosphate buffer (DBA 0.5 mg/ml, hydrogen dioxide 0.03%). The preparations are dehydrated and placed into synthetic resin under cover slides (Entellan, Merck).

### II.2.15 Method of Isolation of CD 34+Endothelial Cells from the Preparation of Mobilized Stem Cells

To obtain a cell preparation of endothelial cells (EC), a standard procedure of a stem hematopoietic cells CD34+ CD133+ separation from leukoconcentrate of mobilized peripheral blood and a magnetic beads separation (CD34+) followed by a short-term standard cell culturing was applied. Culture medium consists of Iscove's modified Dulbecco's medium with added 0.5 g/l of serum albumin, 0.39 μg/ml of human insulin and 60 μg/ml of transferrin. 100 ng/ml of stem cell factor (SCF), 20 ng/ml of Flt3 (Flt3L) and 20 ng/ml of thrombopoietin (TPO) are added to the culture medium. Culturing regimen is the following: the cells are cultured at 37° C. in the atmosphere of 5% CO₂, and fresh medium and cytokines are added every three days. Period of culturing makes 5 to 7 days.

To avoid contamination of the cell preparation by autologous mononuclears, erythrocytes, thrombocytes and other blood elements, the leukoconcentrate is purified therefrom. The preparation of purified mononuclears is prepared from the cryopreserved and unfrozen cell preparation of mobilized autologous stem cells (MASC) by outwashing 10% DMSO solution therefrom by means of triple centrifuging at 2,000 rev/min with physiological solution of 0.9% NaCl.

Stage III. Reconstructive Neurosurgical Operation on the Spinal Cord or Brain, Intraoperative Preparation of ACBP NEPS, Implantation of ACBP NEPS

### III.1 Intraoperative Preparation of ACBP NEPS

The cell biocomposition is prepared in sterile conditions either directly in a surgical room (ex tempore) or in advance in a culture laboratory (to be implanted within 6 hours). The amount of the ACBP NEPS is defined depending on the volume, type and location of the injury. Provisional calculation of necessary amount of the ACBP NEPS is performed according to MRI or CT imaging results of the brain or spinal cord (BSC). The ACBP NEPS is prepared as follows. The cell preparations in the above-mentioned proportions are mixed in a sterile tube in required volumes and allowed to stir by means of 2 to 3 syringe intakes from and discharges back into the tube. The Sphero®GEL preparation is taken out of a refrigerator and warmed to 37.5 to 38° C. A disposable syringe of the volume corresponding to the required calculated volume of the ACBP NEPS is used. The plunger is removed from the syringe and the needle cannula is closed with a rubber or plastic plug (the syringe is used as a tube). 1 ml of the Sphero®GEL is placed on the bottom of the syringe tube and up to 1 ml of the cell biocomposition is added. The tube is centrifuged for 2 minutes in a minicentrifuge at 2,000 rev/min. Liquid component of the biocomposition with the cells is infused into the Sphero®GEL during centrifuging. If a larger volume of the ACBP NEPS is to be prepared, the above mentioned procedure is performed in layers in the syringe tube by placing the Sphero®GEL and the cell biocomposition alternately in layers as in a sandwich and then centrifuged for 2 to 3 minutes. The plunger is returned into the syringe and the air is removed therefrom. A plastic sterile venous catheter is put on the syringe cannula, which catheter will be used for implantation. Within 30 to 40 minutes after preparation of the ACBP NEPS, it is left in the syringe under sterile conditions at room temperature (20 to 22° C.) to cool down and to acquire toothpaste consistence. Then the ACBP NEPS can be implanted.

### III.2 Reconstructive Surgery on the Brain or Spinal Cord and ACBP NEPS Implantation

The plasty of SC defects are reconstructed with the use of the ACBP NEPS in a standard neurosurgical operation of decompressive laminectomy on the level of injury by opening dura mater (DM) and performing radiculomyelolysis of the site of injury. The ACBP NEPS is placed on the surface of spinal cord defect and modeled along the injury. In case of intramedullar or intracerebral implantation, the ACBP NEPS is isolated from direct impact action of CSF. Hence, an intramedullar cyst (cysts) of the SC is opened, drained and the ACBP NEPS is implanted inside the SC cyst. In case of neurotmesis of the SC, a conduit is microsurgically modeled in the gap area of the spinal cord, that is a tube-like conductor (TLC) the walls of which are formed either from the inner leaf of the DM of the patient or a hip muscle fascia of the patient or artificial arterial (aortal) graft attached to the DM of the patient. The ACBP NEPS is implanted inside the TLC. Proximal and distal ends of the injured spinal cord are attached (tunneled and sutured to the DM) to the formed conduit.

The SC plasty being completed, the DM is closed and sutured with the inner leaf of the autologous DM or an artificial DM, then the site of the DM plasty is covered with a standard biopolymer glue (like Tissucol®). The implantation of the ACBP NEPS into brain defects should be done after evacuation of the content (CSF, detritus, etc) out of cystic cavities of the brain during open neurosurgical operations, as well as in the course of functional neurosurgical interventions (stereotaxis, endoscopic manipulations, etc.) or the implantation of the ACBP NEPS in vegetative ganglia. It was shown that the ACBP NEPS degrades within 12 to 24 months, and the site of the prosthesis is transubstantiated, partially or completely, by fibers of autologous nervous tissue. Subsequent post-interventional neurovisualization of the BSC plasty area with the use of MRI imaging 8 to 12 months after the intervention allows for verification and evaluation of the reconstructed BSC tissue structure morphology.

After the ACBP NEPS implantation, the patient is under observation for 10 days 24 hours a day. An intensivist together with an attending doctor should control the condition of the patient to assess complication risks. Besides, regular observation by a neurosurgeon and a neurologist is recommended, who should work in close cooperation with hematologists, transplantologists, immunologists and laboratory specialists.

The main efficacy criteria of this intervention are improvement of neurological symptoms (motor, sensation and bowel and bladder functions). The period of first manifestation of expected results is highly individual and depends on the scope of the BSC injury, the length of the post-injury period, level of compensation of injured functions. Therapeutical efficiency varies from 7 days to 48 months after tissue engineering surgery and is evaluated by the indexes of ASIA (American Spinal Injury Association), FIM (Functional Independence Measurement) and neurophysiological methods of testing (cerebral EEG mapping, transcranial magnetic stimulation, somatosensory evoked potentials, electroneuromyography and complex urodynamic testing).

### INDUSTRIAL APPLICABILITY

Efficiency of the present invention was evaluated in the patients with consequences of brain and spinal cord injuries in comparison with conventional surgical treatment. To objectify received clinical results, specific ASIA and FIM indexes were involved, as well as data received by MRI, electroneuromyography, cerebral EEG mapping, complex urodynamic testing, immunochemical tests of blood and CSF. The trial was done in 50 patients. Surgeries in tissue engineering with the ACBP NEPS implantation were given to 30 patients with severe traumatic disease of the spinal cord. All the patients enrolled into the trial were subdivided into two groups. The 1^{st} group (main, 30 patients) consisted of the patients who had been operated with the ACBP NEPS implantation, the 2^{nd} group (control, 20 patients) included patients that received conventional surgical treatment (decompressive laminectomy, microsurgical radiculomyelolysis, drainage of intramedullar cysts, DM plasty). Distribution of the patients by age and gender is shown in Table 1.

TABLE 1
Distribution of patients by age and gender

| | 1^{st} group | | | | 2^{nd} group (control) | | | |
|---|---|---|---|---|---|---|---|---|
| | Male | | Female | | Male | | Female | |
| Age | Num- | | Num- | | Num- | | Num- | |
| (years) | ber | % | ber | % | ber | % | ber | % |
| 14-19 | 2 | 9 | 1 | 12.5 | 3 | 20 | — | — |
| 20-30 | 11 | 50 | 2 | 25 | 5 | 33.3 | 5 | 100 |
| 31-40 | 7 | 31.8 | 4 | 50 | 6 | 40 | — | — |
| 41-50 | 1 | 4.5 | 1 | 12.5 | 1 | 6.7 | — | — |
| 51-60 | 1 | 4.5 | — | — | — | — | — | — |
| Total | 22 | 100 | 8 | 100 | 15 | 100 | 5 | 100 |

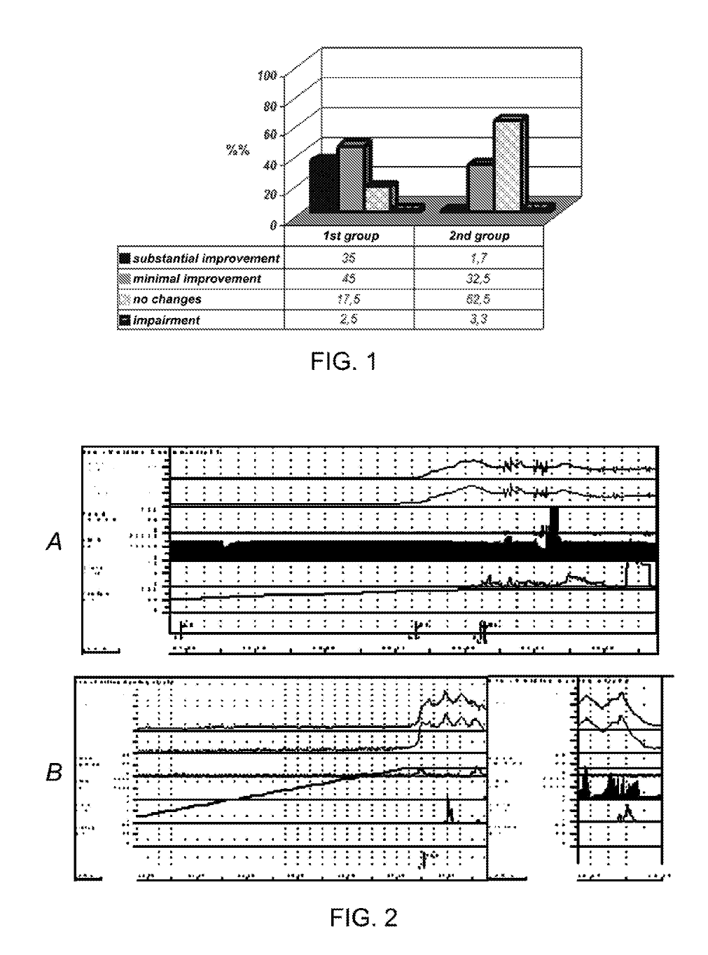

Comparison of efficiency of SC tissue engineering by the ACBP NEPS implantation for traumatic disease of SC and brain with conventional surgical therapies is demonstrated in FIG. 1. The 1^{st} group shown here presents the patients that received surgical treatment with the ACBP NEPS implantation according to the present invention; the 2^{nd} group is the patients of the control group that received conventional surgical interventions. Efficiency of SC tissue engineering with the ACBP NEPS implantation for the SC injury (SCl) was statistically significant (p<0.05) as compared to the control group. Significance (p<0.05) was calculated by χ² method.

FIG. 2 shows changes of urodynamic values in patient V before and after the ACBP NEPS implantation according to the present invention (A—urogram before the therapy, B—urogram after the ACBP NEPS implantation).

The received data are also shown in Tables 2 and 3 and accompanied by clinical Examples 1 and 2.

TABLE 2
Results of therapy of SCI patients after tissue engineering surgeries with ACBP NEPS implantation.

| | | | Time | ASIA, FIM | | | ASIA, FIM | | Time | | | Bowel |
|---|---|---|---|---|---|---|---|---|---|---|---|---|
| | | | after | indexes | | ACBP NEPS | indexes | | after | | | and |
| | | Injury | injury | before | | compo- | after | | treatment | | Motor | bladder |
| No | Patient | level | (years) | therapy | | sition* | treatment | | (months) | Sensation | restoration | control |
| 1 | B-ev N. V. | C5 | 4 | A | 46 | No. 2 | B | 48 | 11 | T1 | Complete | Partial |
| 2 | P-us M. | T7 | 2 | A | 46 | No. 4 | A | 46 | 10 | T10 | Partial | Partial |
| 3 | Sch-ba F. | C7 | 8 | A | 37 | No. 3 | A | 37 | 11 | T3 | Partial | Partial |
| 4 | T-syan A. | T1 | 2 | A | 24 | No. 3 | A | 24 | 11 | T5 | Partial | Partial |
| 5 | R-ska D. | T11 | 2 | A | 31 | No. 4 | C | 37 | 10 | Complete | Partial | Complete |
| 6 | K-kin A. V. | T10 | 3 | A | 46 | No. 2 | B | 46 | 10 | L2 | Partial | Complete |
| 7 | Al Azzi D-ll | C5 | 3 | A | 57 | No. 4 | A | 57 | 8 | C7 | Partial | Complete |
| 8 | N-shin A. A. | C3 | 7 | A | 30 | No. 2 | A | 32 | 8 | C7 | Partial | Complete |
| 9 | M-t A. | T10 | 2 | A | 18 | No. 4 | A | 18 | 6 | T12 | No | No |
| 10 | D-es P. | C5 | 3 | A | 49 | No. 4 | A | 49 | 1 | T6 | Partial | No |
| 11 | A-yan E. | T5 | 5 | A | 24 | No. 4 | A | 16 | 17 | T5 | Partial | Complete |
| 12 | B-chuk N. N. | T12 | 6 | A | 25 | No. 4 | A | 21 | 14 | Complete | Partial | Partial |
| 13 | Sh-lekh N. | T7 | 5 | A | 35 | No. 4 | B | 27 | 30 | T10 | Partial | Partial |
| 14 | H-m N. | T5 | 2 | A | 24 | No. 4 | A | 18 | 12 | T12 | Partial | Complete |
| 15 | D-ov T. | T4 | 5 | A | 22 | No. 3 | B | 17 | 31 | T7 | No | Complete |
| 16 | Ya-in S. A. | C7 | 4 | A | 53 | No. 3 | A | 50 | 30 | T3 | No | Complete |
| 17 | Sh-ga Ya. | T4 | 16 | A | 36 | No. 4 | C | 24 | 27 | Complete | Partial | Complete |
| 18 | Kh-osh P. M. | T5 | 4 | A | 44 | No. 3 | A | 38 | 24 | T7 | Partial | Partial |
| 19 | L-kov A. S. | T5 | 6 | A | 43 | No. 3 | A | 39 | 26 | T8 | Partial | Partial |
| 20 | A-ev Ye. L. | T6 | 2 | A | 31 | No. 3 | A | 27 | 26 | T10 | Partial | Complete |
| 21 | S-ds E. | C7 | 5 | A | 46 | No. 2 | B | 44 | 15 | C7 | No | Partial |
| 22 | V-ev A. V. | T2 | 2 | A | 44 | No. 2 | B | 37 | 25 | T2 | Partial | Partial |
| 23 | V-der L. | C5 | 2 | A | 66 | No. 3 | A | 66 | 25 | C7 | Partial | Partial |
| 24 | I-va O. A. | T3 | 5 | B | 54 | No. 4 | C | 39 | 25 | T6 | Complete | Complete |
| 25 | Sch-kov S. G. | T8 | 11 | B | 35 | No. 1 | C | 34 | 45 | T9 | Partial | Partial |
| 26 | O-kov A. N. | T7 | 10 | A | 28 | No. 3 | B | 27 | 44 | T12 | Partial | Complete |
| 27 | L-shin P. A. | C6 | 1 | A | 62 | No. 3 | C | 52 | 42 | T4 | Partial | Partial |
| 28 | L-va O. D. | T8 | 6 | A | 27 | No. 3 | B | 15 | 42 | T10 | Partial | Complete |
| 29 | Yu-va O. V. | T12 | 1 | A | 24 | No. 2 | B | 14 | 41 | L2 | Partial | Complete |
| 30 | V-na N. V. | L2 | 4 | A | 19 | No. 1 | A | 19 | 41 | L2 | No | No |

*ACBP NEPS composition:
No. 1: Sphero ®GEL; No. 2: Sphero ®GEL, MN and EC; No. 3: Sphero ®GEL, NGEC and NSC; No. 4: Sphero ®GEL, NSC, NGEC, MN and EC

TABLE 3
Changes in neurological symptoms in some SCI patients
after tissue engineering with ACBP NEPS implantation.

| | Time after | |
|---|---|---|
| | surgery | |
| Patient | (months) | Neurological progress |
| V-na | 36 | No neurological progress. After removal of metal construction, |
| | | the spine became unstable. |
| O-kov | 24 | Deep and touch sensation improved. Movement appeared in left |
| | | toes. Full control of bladder and bowel, and sexual function |
| | | restored. |
| Sch-kov | 32 | No significant clinical effect was observed. |
| Yu-va | 19 | Able to stand, to “fix” knees, to walk 12 steps. Bowel and |
| | | bladder control restored, deep and touch sensation improved. |
| L-va | 15 | Level of sensation moved 25 cm down. Able to move legs when |
| | | asked, control bladder and bowel. Able to make 15 backwards |
| | | and 2 to 3 steps forward. |
| D-ov | 6 | Deep sensation restored, as well as function of bowel and |
| | | bladder. Able to cycle on a stationary bike without assistance. |
| Ya-kin | 5 | Weakness in right arm increased, then restored in 7 months, |
| | | mosaic improvement of sensation |
| V-ev | 4 | Right arm movement improved, sensation in back and buttocks |
| | | area restored, as well as sexual function. |
| L-kov | 5 | Controls bowels and anus, restored thermal regulation and |
| | | sweating in lower extremities. Restored movement in toes. |
| A-ev | 7 | Controls movement of both legs, the elements of deep sensation |
| | | appeared. |
| Sh-ga | 8 | Controlled movement in right leg appeared, no pains in the site |
| | | of injury. Thermal regulation and sweating fully restored, as well |
| | | as bowel and bladder function. Able to cycle on a stationary bike |
| | | without assistance. |
| I-va | 6 | Knee (S < D) and Achilles(S < D) reflexes developed, leg |
| | | spasticity increased, bowel and bladder functions fully restored. |
| L-shin | 20 | Able to turn in his bed without assistance, to sit. Epicystostoma |
| | | is removed. Arm movement has improved by 50%, bowel and |
| | | bladder functions restored. Able to eat, drink, brush teeth, and |
| | | serve himself without assistance. |

Hence, methods of tissue engineering with the ACBP NEPS implantation according to present invention for severe traumatic injuries of brain and spinal cord (including completely severed spinal cord) can be considered as the method of choice, the efficiency of which reaches 45% in the cases when all traditional methods and treatment approaches are practically inefficient. Conventional surgical approach to the treatment of severe repercussions of BSC traumatic injuries does not result in significant improvement of neurological symptoms, while application of the proposed medical technique considerably improves life quality of the patients with severe SCl and brain injury and significantly ameliorates functional and social independence of the patients (see Tables 2, 3).

To date, the proposed method of neurosurgical operation is the only efficient therapy of patients with repercussions of severe BSC traumatic injuries, including complete anatomical neurotmesis of the spinal cord (with the gap up to 5 cm) and can lead to significant improvement of their condition, life quality as well as better social adaptation. Possible return of the patients after brain and/or spinal cord injury to normal social activity results in higher economical efficiency considering both return to work activity and reduction of expenses for long-term and inefficient treatment.

### Clinical Example No. 1

Patient Sch-ga, 50. Complaints: steady agonizing girdle pains on the level of left costal arch, absence of voluntary movements in the legs, absence of all types of sensation from the level of inguinal fold, constipation and no controlled urination (catheterizes herself for 14 years).

The patient received the injury on thoracic level of the spinal cord in road accident in 1991: complicated compression fracture of ThIII and ThIV vertebrae. In early period lower paraplegia was observed, lower para-anesthesia from the level of the Th15 segment. No surgery was given to thoracic level of spine. The patient has repeatedly received specialized training in rehabilitation centers with no notable effect.

The patient was admitted to Neurovita Clinic on Oct. 2, 2006 to be enrolled into the research program “New Cell Technologies to the Medicine”. At the admission time the condition of the patient was compensated. Skin and visible mucosa were clean, of normal color, wet. Regional lymph nodes were not palpated. Chest was of regular shape. Breathing was independent, adequate, and free. Respiratory rate was 16 per minute. Pulse was rhythmical, of a satisfactory quality, 88 per minute. Blood pressure was 120/60. Heart tones were clear, rhythmical. The tongue was pink, wet. Abdomen was soft, painless. The liver was not enlarged, spleen was not palpated. The kidneys were not palpated in the seated position. Sense organs and endocrine glands demonstrated no rough defects. Urination was performed with the help of Foley catheter. Defecation was regular every other day with laxative suppositories. The consciousness was clear. The patient was time, space and personality oriented. Pupils were round, D=S, photoreaction was intact, symmetric. Ocular motility was normal, no nistagmus. No face sensation disorders. The face was symmetric; soft palate was symmetric, moving. Swallowing and phonation was intact. No atrophies, pareses, myofacsiculations of trapezius and nodding muscles. The tongue was on the median line, no atrophies, no fasciculations. Coordination tests were performed satisfactorily. Muscle tone in proximal and distal muscle groups was not changed. No hand muscle hypotrophy. Tendon and periosteal reflexes were moderate, no significant difference between the sides. Arm muscle force scored 5. Abdominal reflexes are not evoked. Muscle tone of leg proximal and distal muscle groups was elevated according to spastic type, 3 points by Ashworth index. Notable hypotrophy of calf muscle was observed. Tendon and periosteal reflexes were brisk, D=S. Pathological reflexes of feet, feet and kneecap clonuses were observed. Movements of hip flexor muscles were intact (1 point), as well as of knee extensor muscle (2 points). Pain and temperature para-anesthesia from the level of ThXII, tactile para-anesthesia from the ThIV level. Bowel and bladder dysfunction manifested in constipations and urine incontinence. No meningeal syndrome. Functional evaluation by ASIA index: 56*42*46, level of spinal cord injury—A (complete). Functional evaluation by FIM index was 36%.

The results of complex examination of the patient permitted her inclusion into the research program “New Cell Technologies to Medicine”. The patient received the course of MASC transfusions into subarachnoid space according to the program. The received therapy led to positive effect manifested in the sensation of bladder filling.

The patient received surgical intervention on Sep. 26, 2005 in NeuroVita Clinic: ThII-ThIII-ThIV laminectomy, meningoradiculomyelolysis, tissue engineering of spinal cord with the application of the collagen containing heterogeneous matrix Sphero®GEL with implanted autologous ensheathing glia-olfactory cells (2.8·10⁶), bypassing of the spinal canal with frame-mounted vascular prosthesis <<Gore Tex<<. Postsurgical period was normal, the wound healed with primary adhesion.

Control examination in two years after the surgery demonstrated compensated condition of the patient. Skin and visible mucosa were clean, of normal color, wet. Regional lymph nodes were not palpated. Chest of regular shape. Breathing was independent, adequate, and free. Respiratory rate was 14 per minute. Pulse was rhythmical, of a satisfactory quality, 86 per minute. Blood pressure was 110/70. Heart tones were clear, rhythmical. The tongue was pink, wet. Abdomen was soft, painless. The liver was not enlarged, spleen was not palpated. The kidneys were not palpated in the seated position. Sense organs and endocrine glands demonstrate no rough defects. Urination was performed with intermittent catheterization. Defecation was regular every other day with laxative suppositories. The consciousness was clear, time, space and personality oriented. Pupils were round, D=S, photoreaction was intact, symmetric. Ocular motility was normal, no nistagmus. No face sensation disorders. The face was symmetric; soft palate was symmetric, moving. Swallowing and phonation was intact. No atrophies, pareses, myofacsiculations of trapezius and nodding muscles. The tongue was on the median line, no atrophies, no fasciculations. Coordination tests were performed satisfactorily. Muscle tone in proximal and distal muscle groups was not changed. No hand muscle hypotrophy. Tendon and periosteal reflexes were moderate, no significant difference between the sides. Arm muscle force scores 5. Abdominal reflexes were low, D=S. Muscle tone of leg proximal and distal muscle groups was elevated according to spastic type, 2 points by Ashworth index. Moderate hypotrophy of calf muscles was observed. Tendon and periosteal reflexes were moderate, D=S, No pathological reflexes no clonuses were observed. Hip flexor muscles scored 3 points, knee extensors—2 points, dorsal feet flexors score 2 points, extensors of the 1^{st} toe—2, toe flexors—2 points. The patient can “fix” the knees during verticalization. Pain and temperature para-anesthesia from the level of L2, tactile para-anesthesia from the L3 level. Sensation of bladder feeling developed, as well as voluntary control of urination. The patient does not use catheterization or pampers. No meningeal syndrome. Functional evaluation by ASIA index: 74*98*76, level of spinal cord injury—C (incomplete). Functional evaluation by FIM index was 27%. The results of neurophysiological test showed low-amplitude, unclearly structured cerebral somatosensory evoked potentials during stimulation of both lower extremities. The amplitude of M-response from lower extremities muscles increased.

### Clinical Example No. 2

Patient R-ska, age 37. Complaints: absence of voluntary movements in the legs, absence of all types of sensation from the level of inguinal fold, stool and urinary retention.

The patient suffered an injury on the thoracic level of the spinal cord in a road accident in 2005: a complicated compression fracture of ThXI and ThXII vertebrae. In early period, lower paraplegia was observed, lower para-anesthesia from the Th11 segment.

Underwent 3 surgical interventions: 1. Decompressive laminectomy, transpedicular stabilization on the level of ThX-LII (November 2005, Krakow, Poland); 2. Interbody fusion from anterio-lateral acces using mesh and fixing plate on the level of ThXI-ThXII (January, 2006, Tarnow, Poland); 3. Dismantling of transpedicular system and installation of rigid transpedicular stabilization on the level of ThX-LI-LII (February, 2006, Tarnow, Poland). The patient repeatedly received rehabilitative treatment in reconditioning centers of Poland and Germany with no significant effect.

The patient was admitted to Neurovita Clinic on Oct. 2, 2006 to be enrolled into the research program “New Cell Technologies to Medicine”. At the admission time, the condition of the patient was compensated. Skin and visible mucosa were clean, of normal color, wet. Regional lymph nodes were not palpated. Chest regularly shaped. Breathing was independent, adequate, and free. Respiratory rate was 18 per minute. Pulse is rhythmical, of a satisfactory quality, 82 per minute. Blood pressure was 110/70. Heart tones were clear, rhythmical. The tongue was pink, wet. Abdomen was soft, painless. The liver was not enlarged, spleen was not palpable. The kidneys were not palpable in the seated position. Sense organs and endocrine glands demonstrated no rough defects. Urination was performed with the help of Foley catheter. Defecation was regular every 2 to 3 day with laxative suppositories. The consciousness was clear. The patient was time, space and personality oriented. Pupils were round, D=S, photoreaction was intact, symmetric. Ocular motility was normal, no nistagmus. No face sensation disorders. The face was symmetric, soft palate was symmetric, moving. Swallowing and phonation were intact. No atrophies, pareses, myofacsiculations of trapezius and nodding muscles. The tongue was on the median line, no atrophies, no fasciculation. Coordination tests were performed satisfactorily. Muscle tone in proximal and distal muscle groups was not changed. No hand muscle hypotrophy. Tendon and periosteal reflexes were moderate, no significant difference between the sides. Arm muscle force scores 5. Abdomen reflexes were absent. Muscle tone of leg proximal and distal muscle groups was elevated according to spastic type, 3 points by Ashworth index. Notable hypotrophy of calf muscle was observed. Tendon and periosteal reflexes were brisk, with expanded reflexogenic zones, D=S. Pathological reflexes of feet, feet and kneecap clonuses were observed. Lower spastic paraplegia. Pain and temperature para-anesthesia from the level of LI, tactile para-anesthesia from the LI level. Bowel and bladder dysfunction manifested in the stool and urine retention. No meningeal syndrome. Functional evaluation by ASIA index: 50*74*74, level of spinal cord injury—A (complete). Functional evaluation by FIM index was 31%.

The results of complex examination of the patient permitted her inclusion into the research program “New Cell Technologies to Medicine”. The patient received the course of transfusions of MASC into subarachnoid space according to the program. Received therapy led to the positive effect manifested in the sensation of bladder filling and urge for urination.

The patient underwent a surgical intervention Mar. 7, 2007 in the NeuroVita Clinic: dismantling of transpedicular stabilizing system in ThX-LI-LII levels. LI laminectomy, meningoradiculomyelolysis, tissue engineering of the spinal cord with the application of the collagen containing heterogeneous matrix Sphero®GEL with implanted autologous ensheathing glia-olfactory cells (3.5.10⁶) and mobilized autologous stem cells (EC and MN). Arachnoid membrane modeling. Dura mater plasty. Stabilizing system on the level of ThX-LI-LII restored. Postsurgical period was normal, the wound healed with primary adhesion.

The patient received individual rehabilitation.

Control examination in a year after surgery demonstrated compensated condition of the patient. Skin and visible mucosa are of normal color, wet. Regional lymph nodes are not palpable. Chest is regularly shaped. Breathing is independent, adequate, and free. Respiratory rate was 16 per minute. Pulse was rhythmical, of a satisfactory quality, 86 per minute. Blood pressure was 120/80. Heart tones were clear, rhythmical. The tongue was pink, wet. Abdomen was soft, painless. The liver was not enlarged, spleen is not palpable. The kidneys were not palpable in the seated position. Sense organs and endocrine glands demonstrate no rough defects. Urination was performed with intermittent catheterization. Defecation was regular, every other day with laxative suppositories. The consciousness was clear, the patient was time, space and personality oriented. Pupils were round, D=S, photoreaction was intact, symmetric. Ocular motility was normal, no nistagmus. No face sensation disorders. The face was symmetric; soft palate was symmetric, moving. Swallowing and phonation were intact. No atrophies, pareses, myofacsiculations of trapezius and nodding muscles. The tongue was on the median line, no atrophies, no fasciculation. Coordination tests were performed satisfactorily. Muscle tone in proximal and distal muscle groups was not changed. No hand muscle hypotrophy. Tendon and periosteal reflexes were moderate, no significant difference between the sides. Arm muscle force scores 5. Abdominal reflexes are absent. Muscle tone of leg proximal and distal muscle groups was elevated according to spastic type, 1 point by Ashworth index. Moderate hypotrophy of calf muscles was observed. Tendon and periosteal reflexes were moderate, D=S, No pathological reflexes no clonuses were observed. Movements in hip flexor muscles developed and scored 3 points, knee extensors scored 3 points, dorsal feet flexors scored 3 points, extensors of the 1^{st} toe—3 points, toe flexors—2 points. The patient could independently raise right leg to the height of 50 to 60 cm and make a semi-circle around limb axe. Pain and temperature para-anesthesia from the level of L2, tactile para-hypestesia from L2 level. Sensation of a full bladder developed No meningeal syndrome. Functional evaluation by ASIA index: 78*94*78, level of spinal cord injury—C (incomplete). Functional evaluation by FIM index was 27%. The results of a neurophysiological test showed increase of the amplitude of activity potential of sensory fibers in lower extremities, increase of excitation speed along sensor fibers from both sides, increase of H-reflex habituation after stimulation from both sides.

### LIST OF THE ABBREVIATIONS USED

ACBP NEPS—artificial cell-biopolymer neuroendoprosthetic system
AHSCPB—autologous hematopoietic stem cells of peripheral blood
BS-BSA—buffered saline with bovine serum albumin CNS—central nervous system
BSC—brain or spinal cord
CSF—cerebrospinal fluid
DIT—direct immunofluorescence test
DM—dura mater
DMEM—Dulbecco's modified Eagle medium
DMSO—dimethyl sulphoxide
EC—endothelial cells
EDTA—ethylenediaminetetraacetic acid
FBS—fetal bovine serum
FGF—fibroblast growth factor
FITC—fluorescein isothyocyanate
Flt3L—haemopoietic growth factor
G-CSF—granulocyte colony-stimulating factor
GFAP—gliofibrillar protein
GLP—Good laboratory practice
HBSS—Hank's B\buffered salt solution
HEPES—4-(2-hydroxyethyl)-1-piperazineethanesulfonic acid
HSC—hematopoietic stem cells
IgG—immunoglobulin
MASC—mobilized autologous stem cells
MCA—monoclonal antibodies
MEM—minimum Eagle medium
MN—mononuclears
NC—nuclear cells
NGEC—neuroglial ensheathing cells
NGF—neural growth factor
NSC—neural stem cells
PBS—phosphate buffer saline
PE—phycoerythrin
PerCP or PCP—peridin chlorophyll
SC—spinal cord
SCF—stem cell factor
SCl—spinal cord injury
SSC—side light scattering
TPO—thrombopoetin
TLC—tube-like conductor
VNS—vegetative nervous system

1-23. (canceled)

24. An implantable neuroendoprosthetic system for transubstantiation of defects of brain, spinal cord and vegetative nervous system in a mammal in reconstructive neurosurgical operations, comprising a combination of a heterogeneous collagen-containing matrix for implantation and a biocomposition of cell preparations of various types of autologous cells of a patient, whereby an elastic cell-biopolymer biologically active mass is made.

25. The implantable system of claim 24 wherein said biocomposition comprises, in a NaCl solution, neural stem cells (NSC), neuroglial ensheathing cells (NGEC), endothelial cells with CD34+ marker (EC) and purified mononuclears (MN) in the following ratios (in parts according to numbers of the cells): 0.8 to 1.2 of NSC; 1.6 to 2.4 of NGEC; 4 to 6 of EC; and 4000 to 6000 of MN.

26. The implantable system of claim 25 wherein 0.5 to 1.3% solution of NaCl is used.

27. The implantable system of claim 24 wherein said heterogeneous collagen-containing matrix includes a composition of a heterogeneous implantable gel Sphero®GEL.

28. The implantable system of claim 26 wherein it contains 10⁶ of NSC; 2.10⁶ of NGEC; 5·10⁶ of EC; and 5·10⁹ of MN in 0.5 to 1 ml of 0.9% NaCl solution per 1 ml of said heterogeneous collagen-containing matrix.

29. The implantable system of claim 25 wherein said biocomposition further comprises stimulators of cell regeneration, nerve growth factors and vascular growth factors.

30. A method of production of an implantable neuroendoprosthetic system for transubstantiation of defects of brain, spinal cord and vegetative nervous system in a mammal in reconstructive neurosurgical operations, comprising the steps of
providing a heterogeneous collagen-containing matrix for implantation,
providing a biocomposition of cell preparations of various types of autologous cells of a patient, and
perfusing said biocomposition into said matrix to thereby obtain an elastic cell-biopolymer biologically active mass.

31. The method of claim 30 wherein said perfusing includes centrifuging.

32. The method of claim 31 wherein said centrifuging is carried out within 1.5 to 2.5 minutes at 1,500 to 2,500 revolutions per minute.

33. The method of claim 30 wherein said biocomposition is prepared by defrosting cryopreserved cell preparations in a water bath at 37 to 40° C. and then washing them at least twice in a physiological NaCl solution.

34. The method of claim 30 wherein said biocomposition comprises placed in NaCl solution neural stem cells (NSC), neuroglial ensheathing cells (NGEC), endothelial cells with CD34+ marker (EC), and purified mononuclears (MN).

35. The method of claim 34 wherein said NSC and NGEC are obtained from an olfactory sheath of a nose of a patient, and said EC and MN are obtained from either a bone marrow of the patient or a patient mobilized autologous stem cell leukoconcentrate obtained by separating patient's peripheral blood following stimulating the patient with a granulocyte colony-stimulating factor.

36. The method of claim 34 wherein said biocomposition further comprises stimulators of tissue regeneration, nerve growth factors and vascular growth factors.

37. The method of claim 30 wherein said production of said implantable neuroendoprosthetic system is carried out in sterile conditions directly in an operation room (ex tempore), or in a culture laboratory.

38. A method of performing a reconstructive neurosurgical operation to replace a defect of neural tissue in brain, spinal cord and/or vegetative nervous system of a mammal, comprising the steps of
providing a heterogeneous collagen-containing matrix for implantation,
providing a biocomposition of cell preparations of various types of autologous cells of a patient,
combining said biocomposition and said matrix whereby a neuroendoprosthetic system in the form of an elastic cell-biopolymer biologically active mass is produced, and
implanting said neuroendoprosthetic system into the defect.

39. The method of claim 38 wherein said biocomposition comprises, in a NaCl solution, neural stem cells (NSC), neuroglial ensheathing cells (NGEC), endothelial cells with CD34+ marker (EC) and purified mononuclears (MN).

40. The method of claim 38 wherein said heterogeneous collagen-containing matrix includes a composition of a heterogeneous implantable gel Sphero®GEL.

41. The method of claim 38 wherein said implanting of said neuroendoprosthetic system is performed by placing said neuroendoprosthetic system into said defect and by filling a whole volume of a cyst or a lesion of the brain and/or spinal cord with said neuroendoprosthetic system.

42. The method of claim 41 further comprising covering said neuroendoprosthetic system with an autologous muscle fascia or an artificial dura mater and/or a biodegradable synthetic polymer coat after placing said neuroendoprosthetic system into said defect, to thereby reduce a contact of said neuroendoprosthetic system with patient's liquor.

43. The method of claim 42 wherein said biodegradable synthetic polymer coat includes an implantable biopolymer membrane ElastoPOB®.

44. The method of claim 38 wherein, in case of neurotmesis of the spinal cord, said implanting neuroendoprosthetic system comprises the steps of:
forming a conduit from an artificial arterial graft,
filling, at least partially, said graft with said neuroendoprosthetic system,
placing said conduit in an area of diastasis between the ends of the injured spinal cord, and
suturing pia mater of distal and proximal ends of the injured spinal cord to conduit walls.

45. The method of claim 44 wherein the length of said artificial arterial graft is selected to be equal to the length of said diastasis, and the width of the graft is selected to be equal to the diameter of the spinal cord in the lesion site.

46. The method of claim 38 wherein in an intramedullar or intracerebral implantation said neuroendoprosthetic system is isolated from a direct impact action of cerebrospinal fluid.
